(19) United States
(12) Patent Application Publication
(10) Pub. No.: US 2024/0290416 A1
(43) Pub. Date: Aug. 29, 2024

(54) MICROBIAL COMMUNITY-SCALE METABOLIC MODELING PREDICTS PERSONALIZED SHORT-CHAIN-FATTY-ACID PRODUCTION PROFILES IN THE HUMAN GUT

(71) Applicant: Institute for Systems Biology, Seattle, WA (US)

(72) Inventors: Nick Bohmann, Seattle, WA (US); Christian Diener, Seattle, WA (US); Sean Gibbons, Seattle, WA (US)

(73) Assignee: Institute for Systems Biology, Seattle, WA (US)

(21) Appl. No.: 18/590,897

(22) Filed: Feb. 28, 2024

Related U.S. Application Data
(60) Provisional application No. 63/487,458, filed on Feb. 28, 2023.

(51) Int. Cl. G16B 5/00; G06F 30/20; G16B 40/20

(52) U.S. Cl. CPC G16B 5/00; G06F 30/20; G16B 40/20

(57) ABSTRACT

Kits, methods, systems and software are provided to predict personalized approaches for increasing butyrate production. Such approaches may include diet and/or supplemental interventions. Simulations may be used to predict—for each of one or more background diet—an extent to which a supplemental intervention may improve predicted butyrate production for an individual (e.g., absolutely or relative to a population corresponding to the diet). Disclosed techniques may then be used to identify one or more select diets/supplemental inventions or to rank or one or more select diets/supplemental inventions for a particular individual.

### CROSS-REFERENCE TO RELATED APPLICATIONS

This application claims the benefit of and priority to U.S. Provisional Application No. 63/487,458, filed on Feb. 28, 2023, which is hereby incorporated by reference in its entirety for all purposes.

### STATEMENT REGARDING FEDERALLY SPONSORED RESEARCH OR DEVELOPMENT

This invention was made with government support under R01DK133468 awarded by the National Institutes of Health. The government has certain rights in the invention.

### BACKGROUND

The human gut microbiota maintains intestinal barrier function, regulates peripheral and systemic inflammation, and breaks down indigestible dietary components and host substrates into a wide range of bioactive compounds. One of the primary mechanisms by which the gut microbiota impacts human health is through the production of small molecules that enter the circulation and are absorbed and transformed by host tissues. Approximately half of the metabolites detected in human blood are predicted to be significantly associated with cross-sectional variation in gut microbiome composition.

Short-chain-fatty-acids (SCFAs) are among the most abundant metabolic byproducts produced by the gut microbiota, largely through the fermentation of indigestible dietary fibers and resistant starches, with acetate, propionate and butyrate being the most abundant SCFAs. Deficits in SCFA production have been repeatedly associated with disease. Therefore, SCFA production is a crucial ecosystem service that the gut microbiota provides to its host, with far-reaching impacts on health.

However, different human gut microbiota provided even with the same exact dietary substrate can show variable SCFA production profiles, and predicting this heterogeneity remains a fundamental challenge to the microbiome field. Measuring SCFA abundances in blood or feces is rarely informative of in situ production rates, due to the volatility of SCFAs, cross-feeding among microbes, and the rapid consumption and transformation of these metabolites by the colonic epithelium. Furthermore, SCFA production fluxes (i.e., the amount of a metabolite produced over a given period of time) within an individual can vary longitudinally, depending upon dietary inputs and the availability of host substrates.

### SUMMARY

In some embodiments, microbial community-scale metabolic models (MCMMs) (which mechanistically account for metabolic interactions between gut microbes, host substrates, and dietary inputs) are used to estimate personalized, context-specific SCFA production profiles. These estimations are assessed in view of population-level data (e.g., that includes one or more distributions of SCFA production).

Statistical modeling and machine learning are used to predict metabolic output from the microbiome for a given individual (e.g., based on specimen, demographic, medical-history, or other data identified for the individual). For example, postprandial blood glucose responses can be predicted by machine-learning algorithms trained on large human cohorts. However, many existing machine-learning methods are limited by the measurements and interventions represented within the training data. Mechanistic models like MCMMs, on the other hand, do not rely on training data and provide causal insights. MCMMs are constructed using existing knowledge bases, including curated genome-scale metabolic models (GEMs) of individual taxa. MCMMs can be limited by the inability to find well-curated GEMs for abundant taxa present in certain samples, and this underrepresentation in GEMs tends to be worse in human populations that are generally underrepresented in microbiome research. Despite this, MCMMs can be powerful, transparent, knowledge-driven tools for predicting community-specific responses to a wide array of interventions or perturbations.

In some embodiments, MCMMs are used to facilitate predicting personalized SCFA production profiles in the context of different potential interventions (e.g., one or more dietary, prebiotic, and/or probiotic interventions).

In some embodiments, a computer-implemented method is provided for simulating growth in a gut microbiome taxon model for determining a supplemental intervention of a subject. Measured taxon and abundance data of a gut microbiome sample of a subject is accessed. A plurality of flux balance analysis (FBA) microbial community-scale metabolic models (MCMMs) of the gut microbiome of the subject and a plurality of genome-scale metabolic models (GEMs) of the measured taxon of the subject are generated, where each MCMM constrained by a different background diet. Each MCMM is constrained by a different background diet, and one or more supplemental interventions comprise: (i) a probiotic intervention comprising a probiotic taxon added to the measured taxon, (ii) a prebiotic intervention comprising a non-digestible substrate promoting growth of a beneficial microorganism added to the background diet, or (iii) a combination thereof. The probiotic intervention is added at one or more different doses to the MCMM to determine a response to the probiotic intervention, and wherein the prebiotic intervention is added at one or more different doses to the background diet to determine a response to the prebiotic intervention. Growth in each of the MCMMs is simulated so as to predict metabolic productions from addition of the one or more supplemental interventions to the different background diets in the subject.

The different background diets may be selected from (i) a high-fiber diet such as a vegan high-fiber diet rich in resistant starch or a standard Mediterranean diet, (ii) a low fiber diet such as a standard European diet or a standard American diet, and (iii) a personalized diet. The supplemental intervention may be the combination of the prebiotic intervention and the probiotic intervention. The one or more supplemental interventions may have been absent in the subject before any addition of the one or more supplemental interventions. The growth may be or may have been simulated at increasing increments of the doses of the one or more supplemental interventions so as to generate a plurality of metabolic productions characterizing a dose escalation of the one or more supplemental interventions. The plurality of metabolic productions may include a metabolic production for short chain fatty acid (SCFA) production comprising, or selected from, butyrate production, propionate production, acetate production, or a combination thereof, and wherein the simulation is configured to classify the subject as a responder, non-responder, or regressor based on simulated SCFA production in response to the background diet, the one or more supplemental intervention, or a combination thereof. The method may further include repeating steps (b) and (c) for a classification comprising, or selected from, the non-responder and the regressor, with one or more additional supplemental interventions, and/or the method may further includer ranking the one or more supplemental interventions according to the different background diets and the predicted metabolic production; and (e) generating a gut health management recommendation comprising part or all of the ranking. The ranking may include a heatmap ranking. Generating the gut health management recommendation may further include: (i) mapping the predicted metabolic production of the subject to metabolic production of a reference population, and optionally, clinical phenotypes associated with metabolic production of the subject, the reference population, or a combination thereof, and (ii) generating a distribution of the metabolic production of the reference population and embedding the predicted metabolic production of the subject into a context of the distribution; and (iii) generating a comparative metric using the predicted metabolic production of the subject and the distribution, wherein the comparative metric represents whether or where the predicted metabolic production of the subject falls within the distribution. The mapping and distribution may include the clinical phenotypes associated with metabolic production of the subject, the reference population, or a combination thereof, and wherein the clinical phenotypes are blood-based clinical labs and health markers. The clinical phenotypes may be and/or may include cardiometabolic and immunological health markers. The cardiometabolic and immunological health markers may be associated with butyrate production having (i) significant positive associations with blood-derived markers comprising, or selected from, adiponectin, chloride, and high density lipoprotein (HDL) cholesterol, and (ii) significant negative associations with C-reactive protein (CRP), low-density lipoprotein (LDL), and/or blood pressure. The cardiometabolic and immunological health markers may be associated with butyrate production comprise, or are selected from, absolute monocytes count, alanine transaminase, arachidonic acid, blood pressure, glucose, high sensitivity CRP, LDL, LDL cholesterol, LDL small particle number, LP-IR scores, mean corpuscular hemoglobin concentration, oxidized LDL, platelets, triglyceride/HDL ratio, triglycerides, uric acid, and/or zinc.

In some embodiments, a gut health intervention identification system comprising one or more processors; and memory coupled to the one or more processors, wherein the memory comprises computer-executable instructions causing the one or more processors to perform part or all of one or more processes and/or part or all of one or more methods disclosed herein. A process or method may include: (a) receiving butyrate production data of: (i) a gut microbiome of a subject simulated on a plurality of different background diets with and without one or more supplemental interventions, the one or more supplemental interventions comprising, or selected from, a prebiotic intervention, a probiotic intervention, or a combination thereof, and (ii) a plurality of gut microbiomes of a reference population comprising generally healthy individuals each individually simulated for butyrate production on essentially the same background diets as the subject, and optionally, essentially the same supplemental interventions as the subject (b) generating, for each of the plurality of different background diets, a distribution based on butyrate production data of the subject and the reference population associated with the background diet; (c) generating, for each of the plurality of different background diets, a comparative metric using the distribution for the background diet and the butyrate production data of the subject simulated on the background diet; and (d) identifying, based on the comparative metrics, a particular intervention to recommend for the subject, where the particular intervention includes a particular background diet, one or more particular supplemental interventions, or a combination thereof.

The particular intervention may be predicted to result in a butyrate production in the subject that meets or exceeds a minimum healthy butyrate production threshold of the reference population simulated for butyrate production on essentially the same background diet as the subject. The minimum healthy butyrate production threshold may be a cutoff between lower and inter quartiles of the reference population for butyrate production. A disclosed process or method may further include generating a gut health report embedding the butyrate production data of the subject into a context of the distribution of the butyrate production of the reference population for a given background diet of the plurality of different background diets, the gut health report identifying the particular intervention. The identifying the particular intervention may comprise ranking the plurality of background diets based on the comparative metrics. The ranking may further comprise identifying a clinical phenotype associated with the ranking, and wherein the process further includes generating a gut health management recommendation based on part or all of the ranking. A process or method may further include generating one or more associations between clinical phenotypes and butyrate production of the reference population, and generating a comparative metric using the associations, wherein the comparative metric represents whether the predicted butyrate production of the subject is positively or negatively associated with the clinical phenotype. The clinical phenotype may include, or may be selected from, cardiometabolic and immunological health markers. The cardiometabolic and immunological health markers may be associated with butyrate production, the butyrate production having (i) significant positive associations with blood-derived markers comprising, or selected from, adiponectin, chloride, and high density lipoprotein (HDL) cholesterol, and (ii) significant negative associations with C-reactive protein (CRP), low-density lipoprotein (LDL), and blood pressure. The cardiometabolic and immunological health markers may be associated with butyrate production comprise: absolute monocytes count, alanine transaminase, arachidonic acid, blood pressure, glucose, high sensitivity CRP, LDL, LDL cholesterol, LDL small particle number, LP-IR scores, mean corpuscular hemoglobin concentration, oxidized LDL, platelets, triglyceride/HDL ratio, triglycerides, uric acid, or zinc. The plurality of background diets may comprise a high-fiber diet such as a vegan high-fiber diet rich in resistant starch or a standard Mediterranean diet, a low fiber diet such as a standard European diet or a standard American diet, and a personalized diet. A disclosed process or method may further include, for each of the plurality of different background diets with and without one or more supplemental interventions: generating a classification that predicts whether the subject will be a responder, non-responder, or regressor to the background diet with or without the one or more supplemental inventions, wherein the responder exhibits essentially an increase in butyrate production, the non-responder exhibits essentially no change in butyrate production, and the regressor exhibits essentially a decrease in butyrate production.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present disclosure is described in conjunction with the appended figures:

FIG. 1 includes panels that (A): represent an in silico medium containing a matched diet mapped to its constituent metabolic components;
(B) depicts a representations of the generation of an MCMM;
(C) demonstrates how the growth in the MCMM simulated through cooperative tradeoff flux balance analysis (ctFBA);
(D) illustrates how predicted levels of SCFA production fluxes are validated; and
(E) illustrates how predicted and measured SCFA production fluxes are compared.

FIG. 2 illustrates predictions of SCFA production using 16S amplicon sequencing or shotgun metagenomic sequencing data.

FIG. 3 illustrates relationship between predicted and measured butyrate production rates in in vitro and ex vivo cultures.

FIG. 4 illustrates that the divergence in SCFA production between controls and fiber-treated samples is related to culture dilution.

FIG. 5 illustrates the quantitative agreement between human stool ex vivo assays between measured and predicted SCFA production fluxes within an across fiber treatment groups.

FIG. 6 illustrates that MCMMs built from shotgun metagenomic sequencing data perform better when constructed at the species level as compared to the genus level.

FIG. 7 illustrates that alpha diversity of communities does not account for differences in SCFA.

FIG. 8 demonstrates that predicted SCFA production profiles were associated with variable immune response groups following a high-fiber dietary intervention.

FIG. 9 illustrates that SCFA flux predictions are significantly associated with blood-derived clinical markers.

FIG. 10 illustrates that microbial MCMMs can be used to design and select personalized prebiotic, probiotic, and dietary interventions aimed at optimizing SCFA production profiles.

### DETAILED DESCRIPTION

The various embodiments of the inventions and methods disclosed herein provide a technical solution to the technical problem of estimating small chain fatty acid production in subjects in order to develop subject-specific recommendations in regard to supplemental interventions.

In some embodiments, a kit and/or method is availed to process a fecal sample. The fecal sample is processed to approximate the content of the subject's gut microbiome. It will be appreciated that a variety of techniques and molecular biology approaches may be used to characterize the gut microbiome, including 16S ribosomal RNA (rRNA) amplicon sequencing or shotgun metagenomic sequencing. Any method for characterizing the types and relative quantities of bacteria within the stool sample may be used.

Once the composition of the gut microbiome has been predicted, at least part of the predicted composition may be used to approximate a rate of SCFA production in the subject. The SCFA production rate may be a production rate of all SCFAs or of one or more specific SCFAs (such as propionate, acetate, and/or butyrate). Converting the composition of the gut microbiome data to a prediction SCFA production rate may be performed by transforming the at least part of the predicted gut-microbiome composition using mathematical model, such as an MCMM.

An MCMM is used to understand, predict, and manipulate the metabolic interactions within microbial environments. An MCMM is particularly useful in studying complex microbial ecosystems, such as those found in the human gut, soil, and water environments, where multiple species of microorganisms coexist and interact with each other and their environment. MCMMs are based on the principles of systems biology and metabolic engineering. They extend the concept of genome-scale metabolic models, which are used to represent the metabolic capabilities of a single organism, to the community level. By integrating the metabolic networks of multiple species, MCMMs can capture the metabolic interactions between different microbes, including competition for resources, syntrophy (mutualistic relationships where the metabolic byproducts of one organism serve as nutrients for another), and other ecological dynamics.

An MCMM is generated by determining parameters using genomic, transcriptomic, proteomic, and/or metabolomic data for the microbial species present in the community. This data is used for reconstructing the metabolic network of each organism. The MCMM may be generated with data representing the species, genus, or any other taxonomic level depending on the data available. Here, the term genus will be used, but it should be appreciated that MCMMs may be generated using other taxonomic levels. For each genus a metabolic network is constructed, detailing the metabolic pathways and reactions that the organism can perform. This includes the enzymes involved, the substrates and products of each reaction, and the genes encoding these enzymes. The number and detail enzymatic reactions may be varied in MCMMs. Some MCMMs may include the complete known enzymatic pathways for an organism, or only a subset of such pathways. Individual taxa models are then integrated into a single community model. This integration requires consideration of the metabolic interactions between organisms, including nutrient exchange and the impact of environmental conditions on community dynamics.

MCMMs can be analyzed using various computational methods, such as flux balance analysis (FBA) or a derivative of the approach called cooperative tradeoff flux balance analysis (CTFBA), to predict the metabolic fluxes (rates of metabolic reactions) under different conditions. This analysis can reveal insights into the roles of different species within the community, predict the community's response to environmental changes, and identify potential metabolic targets for possible targeted interventions. FBA relies on the stoichiometry of metabolic reactions and constraints such as mass balance, energy conservation, and capacity limits of enzymes (flux capacities) to predict the distribution of fluxes (reaction rates) across the metabolic network that maximizes or minimizes a certain objective function, usually biomass growth or the production of a specific metabolite.

CTFBA extends FBA by considering the cooperative interactions and tradeoffs between different metabolic pathways. In biological systems, metabolic pathways often share intermediates and energy resources, and their activities can be tightly regulated based on the cell's needs. CTFBA aims to capture these complex interactions by modeling cooperative behavior, considering tradeoffs that cells make to balance different objectives, such as maximizing growth while minimizing energy expenditure, and the use of objective functions. Unlike traditional FBA, which typically focuses on a single objective function, CTFBA can incorporate multiple objective functions to reflect the complex optimization problems faced by cells in nature.

As stated above, once the MCMM has been created, a subject's microbiome data may serve as an input into the model and the output of the model may be a predicted SCFA production rate or flux (measured in units such as mmol/L/h).

While the precise SCFA flux is a useful measurement, it is generally accepted that the value of this variable will vary across individual subjects and that the SCFA flux of individual subjects will respond uniquely to dietary interventions. Thus, in some embodiments, the subject specific SCFA flux is considered with respect to other dietary and lifestyle factors, such as the subject's, age, gender, diet, and ethnicity. The subject's specific SCFA flux can then be compared to the distribution of SCFA flux or production rate for subjects of similar demographics and lifestyle. The comparison provides information regarding the feasible range of SCFA production rates that may be obtained given the additional demographic, dietary, and biological constraints.

In some embodiments, the subject's SCFA flux, or the subject's SCFA flux in combination with the distribution of SCFA flux from a population, are used to make recommendations for nutritional supplementation. An objective may be to alter the subject's rate of SCFA production to match the profile of healthier individuals. Typically, higher SCFA production rates, particularly with regards to butyrate, are associated with better health and thus an objective of nutritional supplementation may be to elevate specific SCFA levels. Alternatively, in some embodiments a recommendation for nutritional supplementation may be made with the objective of altering SCFA production rates and biomarkers for disease, such as markers of inflammation that can be measured in routine blood samples. Consistent with such embodiments, the results of a blood test measuring the levels of various biomarkers may be combined with results of the MCMM into a model to may recommendations regarding nutritional supplementation.

The subject's SCFA flux or rate of production may be compared to population data from various sources to generate a comparative metric. The comparative metric represents where the predicted SCFA metabolic production of the subject falls within the distribution. The population data may represent data from subjects with similar demographics or dietary habits, such as a high-fiber or low fiber diet. The dietary data may also represent diets such as a vegan diet, a standard Mediterranean diet, a standard European diet, a standard American diet, or a personalized diet. Such a comparison may reveal the potential for the subject to raise their SCFA metabolic production within their dietary group. For instance, the comparative metric may reveal that a subject has a very low rate of production for their dietary class (i.e. 5th percentile), and thus there is a high likelihood for nutritional supplementation to elevate these levels. Alternatively, if a subject has a high rate of production for their dietary class (i.e. 95th percentile), then the subject may potentially not respond to nutritional supplementary to raise their SCFA flux or rate of production.

The population distribution may also be sourced from data from a population with different dietary habits from the subject. This comparison may generate a comparative metric that demonstrates how the subject altering their diet or other behavior may impact their SCFA metabolic production.

In addition to dietary habits, a subject's SCFA flux or rate of production may also be compared to population distributions for subjects with various blood-measured biometrics, such as cholesterol levels, etc. This comparison may reveal the potential elevations that can be expected within that demographic.

In certain embodiments, the probiotic is a butyrate producing taxon, such as Faecalibacterium. In many embodiments, the Faecalibacterium is added to the taxon model generally at a relative abundance of about 20% or less of the combined total taxon abundance in the MCMM, usually around 15% or less, more typically about 5%-10%.

In various embodiments, the prebiotic is a dietary fiber comprising a resistant starch, a polyphenol, or a combination of a dietary fiber and a polyphenol. A featured example is where the resistant starch comprises, or is selected from, inulin, pectin, and fructo-oligosaccharide. For example, in certain embodiments, the prebiotic is added to the background diet at any relative abundance sufficient to assess response to the probiotic intervention, and generally within 200% of a daily suggested dose. For example, such as about 200 millimoles/gram dry weight*hour (mmol/gDW*h) or less. For example, in many embodiments, the prebiotic is pection, inulin, and/or fructo-oligosaccharide, and is added to the background diet at about 1.0 mmol/gDW*h for pectin, 10.0 mmol/gDW*h for inulin, and 100 mmol/gDW*h for fructo-oligosaccharide.

In some embodiments, the simulating includes solving the MCMM by cooperative tradeoff FBA (ctFBA), and the GEMs are pre-curated.

### Example

An example of an embodiment of the invention is provided below for illustrative purposes. It will be appreciated that some steps are added for clarity even if they are not required and that the omission of a specific step, method, or process does not indicate that it is not a part of the invention.

### MCMMs Capture SCFA Production Rates In Vitro

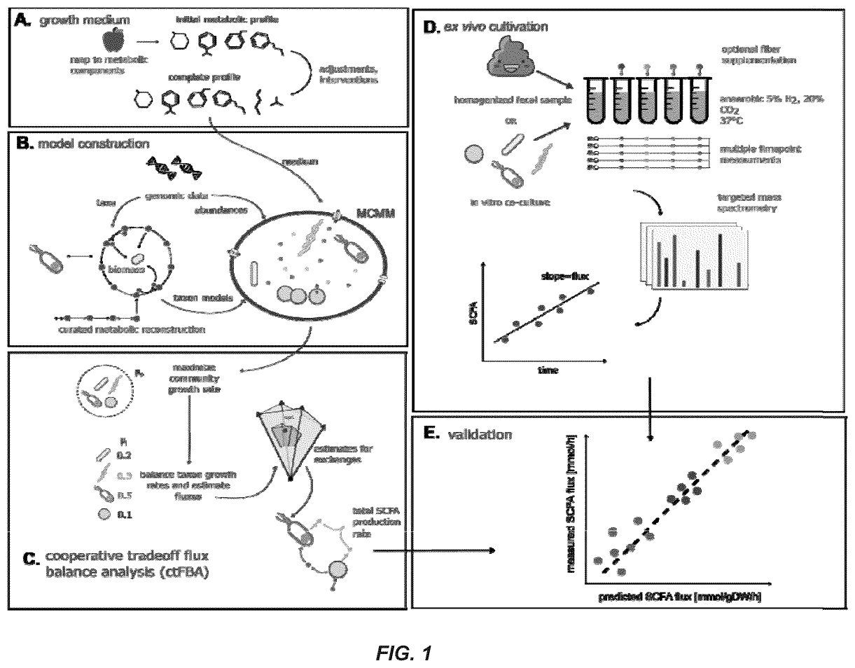

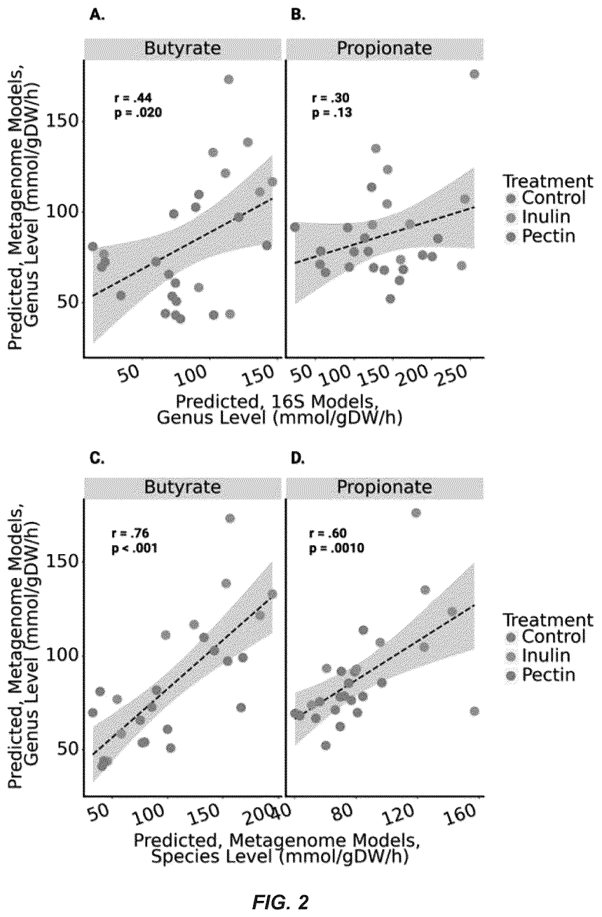

FIG. 1 illustrates how MCMMs may be used to predict production rates of the major SCFAs (i.e., acetate, propionate, and butyrate) under controlled experimental conditions. Growth media, matching the environmental context of each experiment, were constructed and applied as bounds on metabolic import to MCMMs (FIG. 1A), which were concurrently constructed by combining manually-curated GEMs from the AGORA database29 using MICOM21, constraining taxon abundances using 16S amplicon or shotgun metagenomic sequencing relative abundance estimates (FIG. 1B). Sample-specific metabolic models were then solved using cooperative tradeoff flux balance analysis (ctFBA), a two-step quadratic optimization strategy that yields empirically-validated estimates of the steady state growth rates and metabolic uptake and secretion fluxes for each taxon in the model (FIG. 1C, see Materials and Methods below). Models constructed from 16S amplicon sequencing data were summarized at the genus level, which was the finest level of phylogenetic resolution that the data allowed for. When shotgun metagenomic sequencing data were available, models were constructed at the species level. Models constructed from both 16S and shotgun metagenomic data at the species and genus levels showed highly consistent results (FIG. 2). Measured SCFA production profiles from synthetic in vitro community and stool ex vivo experiments (FIG. 1D) were compared to paired SCFA flux predictions from MCMMs to validate the accuracy of the models (FIG. 1E).

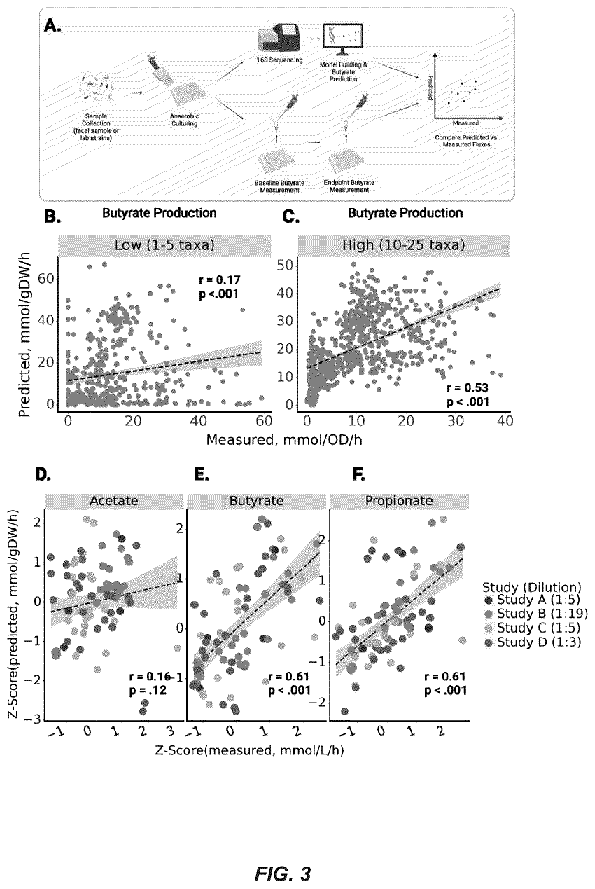

Published data from synthetically constructed communities of bacterial commensals isolated from the human gut30 were initially examined. This data set included endpoint measurements of relative microbial abundances, derived from 16S amplicon sequencing, measured endpoint butyrate concentrations, and the overall optical density for each of 1,387 independent co-cultures (FIG. 3A). Cultures varied in richness from 1-25 strains. MCMMs were constructed for each co-culture as described above, simulating growth of each of the models using a defined medium mapped to a database of metabolic constituents, matching the composition of the medium used in the in vitro experiments (see Materials and Methods below).

Model-predicted butyrate fluxes were compared with calculated butyrate production rates (endpoint butyrate divided by culturing time, assuming no butyrate at the start of growth, normalized to total biomass using OD600), stratifying results into low richness (1-5 genera) and high richness (10-25 genera) communities. Model predictions for butyrate production fluxes were significantly correlated with measured butyrate production fluxes across all communities (Pearson's correlation; Low Richness: r=0.17, p<0.001; High Richness: r=0.53, p<0.001), but the predictions were more accurate in the higher richness communities (FIG. 3B-C).

Next, MCMM predictions were compared to anaerobic ex vivo incubations of human stool samples from a small number of individuals (N=29), cultured after supplementation with sterile PBS buffer or with different dietary fibers across four independent studies. Study A contained samples from two donors cultured for 7 hours with a final dilution of 1:5, Study B18 contained samples from 10 donors cultured for 24 hours diluted 1:19, Study C contained samples from 8 donors cultured for 4 hours diluted 1:5, and Study D contained samples from 9 donors cultured for 6 hours diluted 1:3. Fecal ex vivo assays allow for the direct measurement of bacterial SCFA production fluxes without interference from the host. For all three studies, ex vivo incubations were performed by homogenizing fecal material in sterile buffer under anaerobic conditions, adding control or fiber interventions to replicate fecal slurries, and measuring the resulting SCFA production rates in vitro at 37° C. (see Materials and Methods).

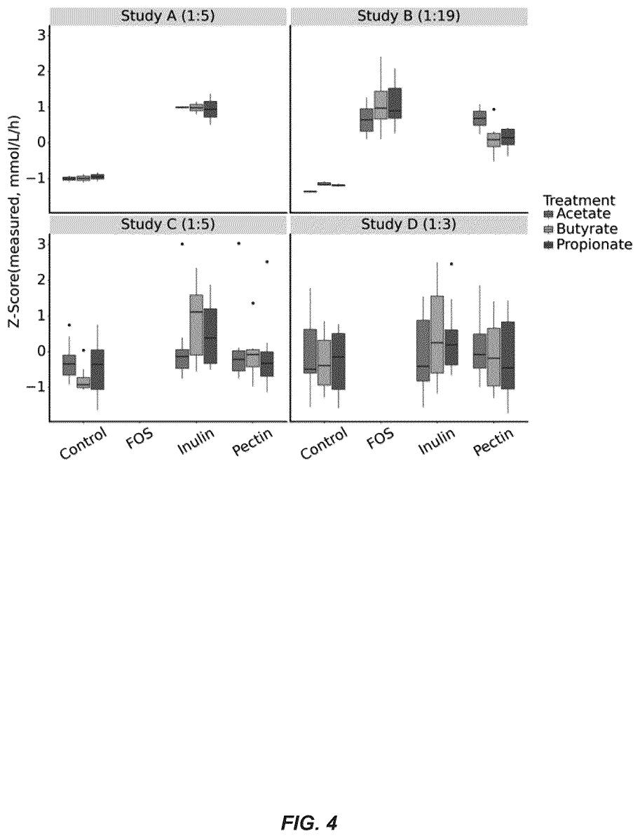

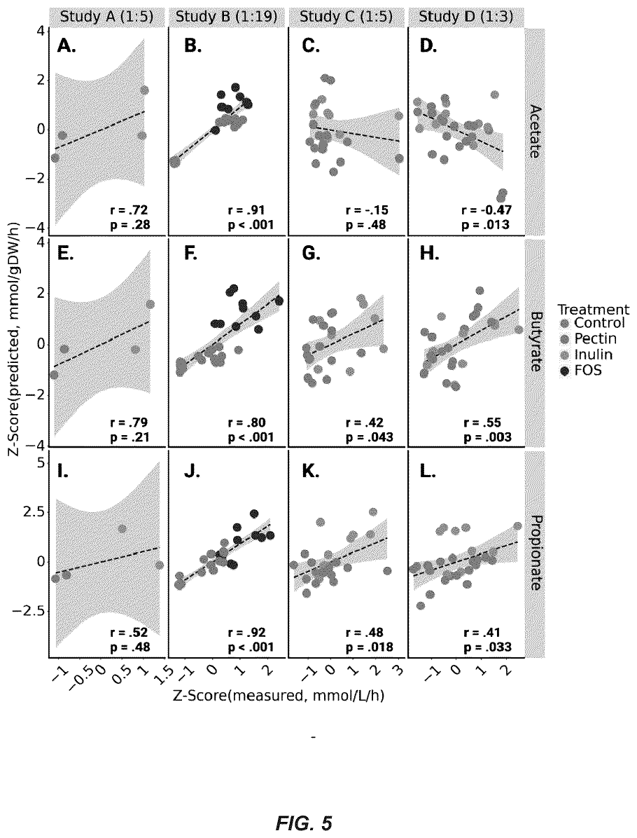

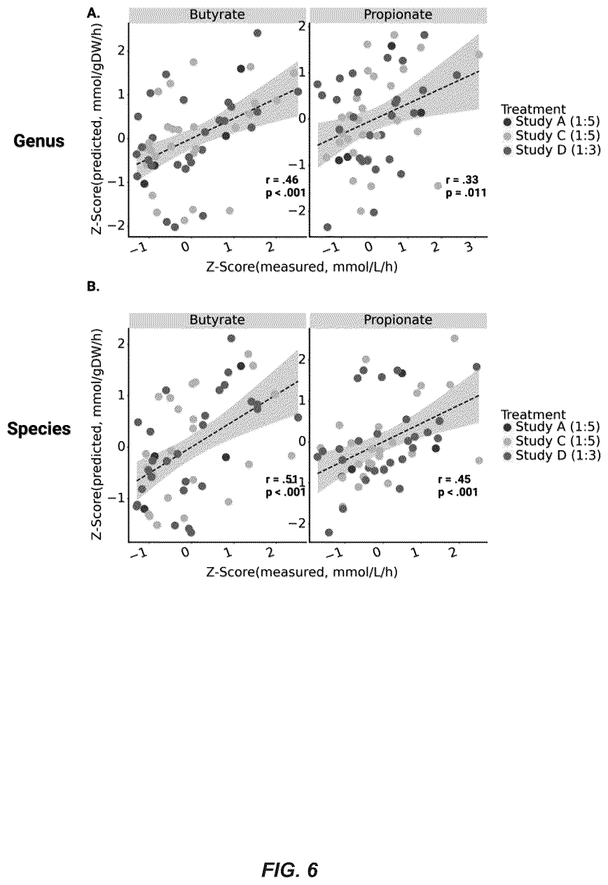

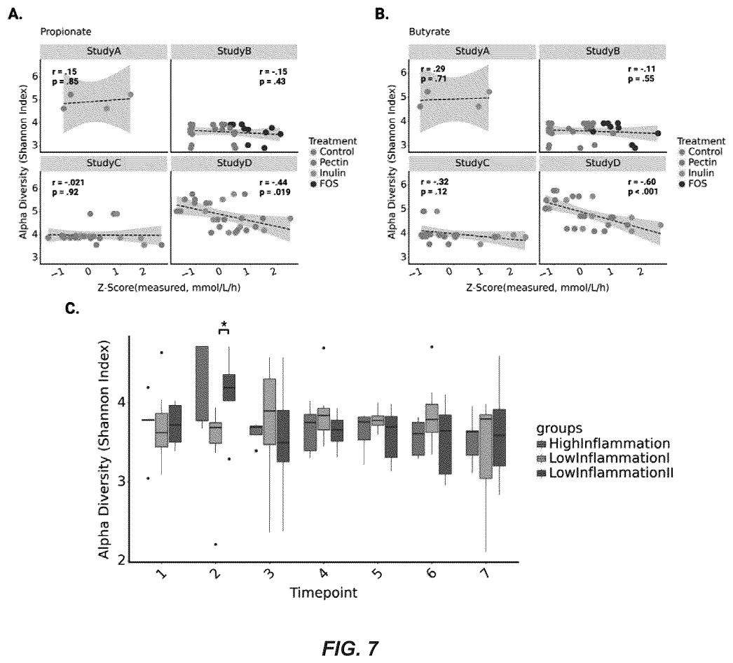

Metagenomic (Studies A, C and D) or 16S amplicon (Study B) sequencing data from these ex vivo cultures were used to construct MCMMs, using relative abundances as a proxy for relative biomass for each bacterial taxon (see Materials and Methods). MCMMs were simulated using a diluted standardized European diet (i.e., to approximate residual dietary substrates still present in the stool slurry), with or without specific fiber amendments, matching the experimental treatments (see Material and Methods below). Within studies, the divergence in measured SCFA production between control samples and fiber-treated samples seemed to be highly dependent upon the final dilution of the ex vivo cultures (FIG. 4). This was accounted for by matching the dilution of residual fiber (starch, cellulose and dextrin) in the medium used for growth simulation to the corresponding study. For instance, Study A was diluted 1:5, so the residual fiber in the medium used to simulate growth for these samples was diluted by a factor of 5. The resulting SCFA flux predictions were then compared to the measured fluxes. MCMM fluxes are given in units of mmol/gDW/h, while measured production fluxes are given in mmol/L/h. Without knowledge of the live-cell biomass within the fecal homogenates, it was not possible to normalize the units across the two axes, but the predicted and measured values were expected to be proportional. To overcome study-specific differences in protocols and allow for comparison of results across studies, both measured and predicted SCFA production fluxes were Z-scored within each data set (FIG. 3D-F). A similar degree of agreement between MCMM-predicted and measured production fluxes for butyrate and propionate was observed across all four ex vivo data sets (FIG. 3E-F). Significant agreement was observed between measured and predicted production fluxes of butyrate and propionate within each individual data set (r=0.41-0.97, Pearson test, p<0.05) with the exception of propionate in Study A, which had a limited sample size (N=2) (FIG. 5E-L). Notably, the correlation coefficient (Pearson r) for these associations was similar to that seen in the high-richness in vitro cultures (FIG. 3C). In studies A and B, acetate production was more readily predicted, likely due to a strong treatment-effect (FIG. 5A-D). Within treatment groups, similar correlations were observed, though statistical power was severely limited by the smaller sample sizes. Predictions from models built with shotgun metagenomic sequencing data showed slightly better results when constructed at the species level, as compared to building at the genus level (FIG. 6). To test whether SCFA production was impacted by sample diversity, butyrate and propionate were measured against Shannon index for each sample in each study (FIG. 7). A weak significant signal was seen in only one of the four studies (Study D). In summary, there was agreement between MCMM predicted and measured in vitro production rates of butyrate and propionate in the presence or absence of fiber supplementation, with better agreement in more diverse communities and over longer experimental incubation times (FIG. 3-3).

MCMM Predictions Correspond with Variable Immunological Responses to a 10-Week High-Fiber Dietary Intervention

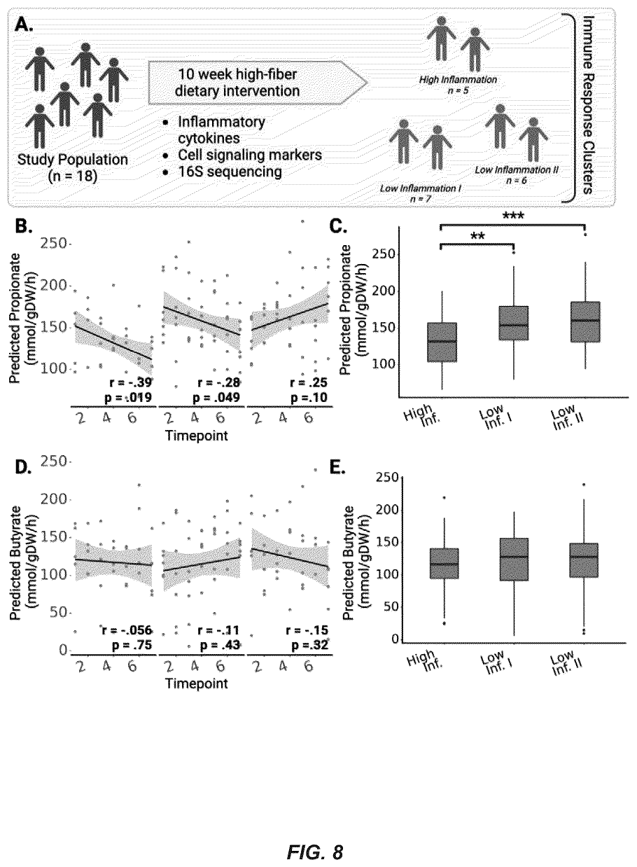

Next, whether MCMM-predicted SCFA production rates could be leveraged to help explain inter-individual differences in phenotypic response following a dietary intervention was investigated. Specifically, data from 18 individuals who were placed on a high-fiber diet for ten weeks was examined. These individuals fell into three distinct immunological response groups: one in which high inflammation was observed over the course of the intervention (high-inflammation group), and two other distinct response groups that both exhibited lower levels of inflammation (low-inflammation groups I and II; FIG. 8A). Immune response groups could be explained, in part, by differences in MCMM-predicted production rates of anti-inflammatory SCFAs. Using 16S amplicon sequencing data from seven time points collected from each of these 18 individuals over the 10-week intervention, MCMMs were built for each study participant at each time point. Growth was then simulated for each model using a standardized high-fiber diet, rich in resistant starch (see Material and Methods). Throughout the study, a trend of decreasing propionate production was observed in high-inflammation individuals (r=0.39, Pearson test, p=0.019), showing less production as the intervention went on, despite the high fiber content of the diets consumed by participants (FIG. 8B). Individuals in the high-inflammation group showed significantly lower predicted propionate production, on average, compared to the individuals in each of the low-inflammation groups (High vs. Low I: 131.9±5.8 vs 158.1±5.7 mmol/(gDW h), Mann-Whitney p=0.0053; High vs. Low II: 131.9±5.8 vs 163.08.3±6.5 mmol/(gDW h), Mann-Whitney p=0.0017; FIG. 8C). Butyrate showed no such significant effects across immune response groups (FIG. 8D, 4E). To investigate whether sample alpha-diversity was sufficient to explain the differences between the immune response groups, the alpha diversity for each sample at each timepoint during the study was calculated. Across all seven time points tested, only one significant difference in alpha diversity was seen, between the two low inflammation groups at time point 2 (Mann-Whitney U-test, p<0.05), suggesting that differences in SCFA production throughout the intervention were not the result of differences in diversity.

MCMM-Predicted SCFA Profiles are Associated with a Wide Range of Blood-Based Clinical Markers

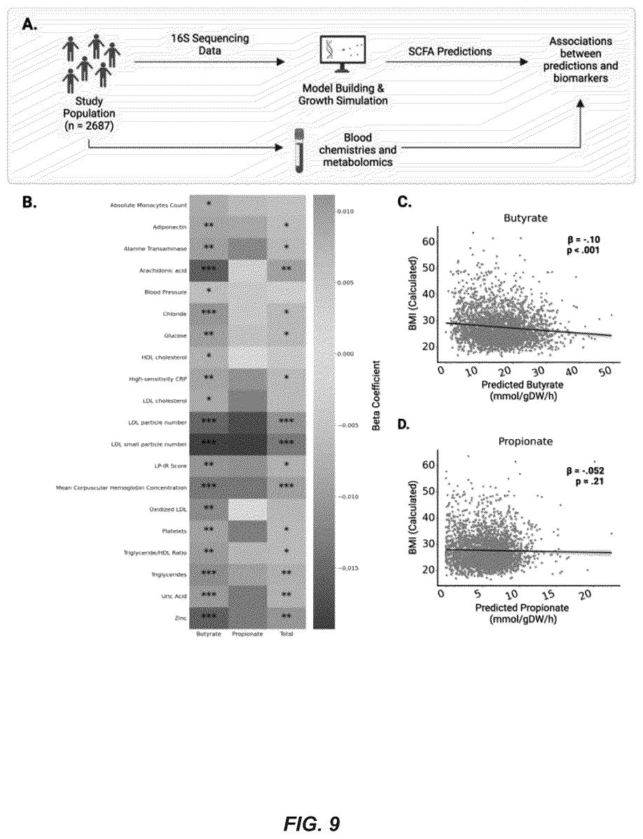

To further evaluate the clinical relevance of personalized MCMMs, SCFA production rate predictions were generated from stool 16S amplicon sequencing data for 2,687 individuals in a deeply phenotyped, generally-healthy cohort from the West Coast of the United States (i.e., the Arivale cohort). Baseline MCMMs were built for each individual assuming the same dietary input (i.e., an average European diet) in order to compare SCFA production rate differences, independent of background dietary variation. MCMM-predicted SCFA fluxes were then regressed against a panel of 128 clinical chemistries and health metrics collected from each individual, adjusting for a standard set of common covariates (i.e., age, sex, and microbiome sequencing vendor; FIG. 9A). After FDR correction, 20 markers were significantly associated with the predicted production rate of butyrate (FIG. 9B). Predicted butyrate production showed significant positive associations with only 3 markers, including the health-associated hormone adiponectin, and significant negative associations with 17 markers linked to disease, including C-reactive protein (CRP), low-density lipoprotein (LDL), and blood pressure (mean arterial pressure; P<0.05, FDR-corrected t-test). Propionate showed no significant associations after covariate adjustment and multiple comparison correction (FIG. 9B). Total combined propionate and butyrate production was significantly associated with 16 clinical markers, all overlapping with those associated with butyrate. Predicted butyrate production was significantly negatively associated with BMI (β=−0.10, t-test, p<0.001), while propionate was not (FIG. 9C-D).

### Leveraging MCMMs to Design Precision Dietary, Prebiotic, and Probiotic Interventions.

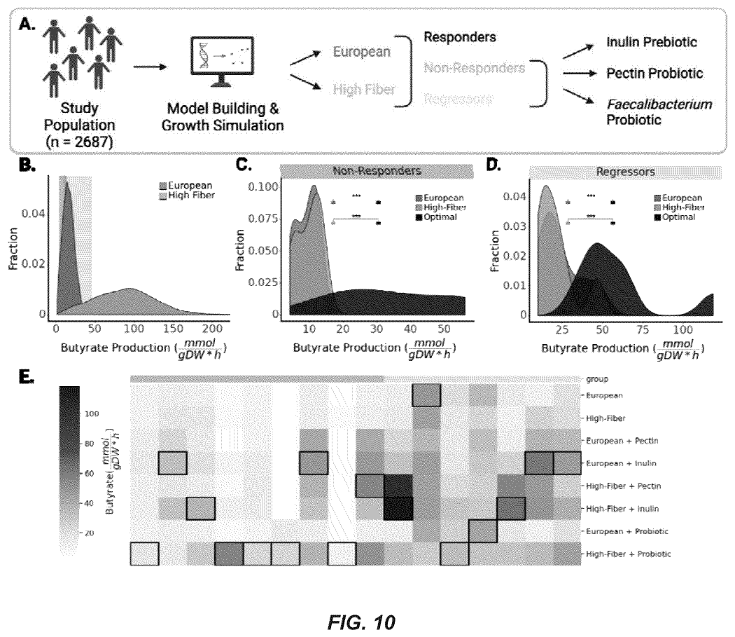

As a proof-of-concept for in silico engineering of the metabolic outputs of the human gut microbiome, a set of potential interventions designed to increase SCFA production for individuals from the Arivale cohort was screened (FIG. 10A). MCMMs were built using two different dietary contexts: an average European diet, and a vegan, high-fiber diet rich in resistant starch (see Material and Methods). As expected, models grown on a high-fiber diet showed higher average predicted butyrate production: 87.78±0.67 mmol/(gDW h) vs 16.29±0.13 mmol/(gDW h), t-test, t=104.3, p<0.001 (FIG. 10B). However, this increase in butyrate production between the European and high-fiber diets was not uniform across individuals. On the high-fiber diet, some individual gut microbiota compositions showed very large increases in butyrate production, some showed little-to-no change, and a small subset of samples actually showed a decrease in butyrate production, relative to the European diet. A set of ‘non-responders’ (N=9) who produced less than 15 of butyrate on the European diet and showed an increase in butyrate production of less than 20% on the high-fiber diet were identified (FIG. 10C). A set of ‘regressors’ (N=7) who showed decreased butyrate production on the high-fiber diet when compared to the European diet was also identified (FIG. 10D). Prebiotic and probiotic interventions were also simulated across these individuals to identify optimal combinatorial interventions for each individual (FIG. 10C-E). MCMMs for each subset of individuals were simulated with prebiotic and probiotic interventions in the context of either the European or the high-fiber dict. Specifically, diets were supplemented with the dietary fiber inulin, with the dietary fiber pectin, or with a simulated probiotic intervention that consisted of introducing 10% relative abundance of the butyrate-producing genus Faecalibacterium to the MCMM. In general, optimal combinatorial interventions significantly increased the population-level butyrate production well above either dietary intervention alone (FIG. 10C-D).

For 15/16 individuals in the regressors or non-responders groups, supplementation of the background diet with a specific prebiotic or probiotic increased the butyrate production rate (FIG. 10C-E). For both regressors and non-responders, the optimal intervention showed substantial increases over the standard European diet (+290±80% for non-responders; +239±102% for regressors). The exact intervention that yielded the highest butyrate production for any given individual across both populations varied widely (FIG. 10E). For example, the probiotic intervention was more successful in raising predictions for butyrate production in non-responders than it was in regressors (FIG. 10E). Overall, no single combinatorial intervention was optimal for every individual in the population.

### Discussion and Conclusion

The objective of this example was to experimentally validate personalized MCMM SCFA predictions. Predictions of butyrate production in synthetically constructed in vitro co-cultures showed significant agreement between measured and predicted butyrate fluxes (FIG. 3).

Further validation of MCMM predictions was observed from ex vivo anaerobic fecal incubations. Strong agreement between SCFA flux predictions and measurements, especially for butyrate and propionate, across four independent studies was observed (FIG. 5). Butyrate and propionate showed a narrow range of possible fluxes for a given biomass optimum, suggesting that the production of these molecules is strongly coupled to biomass production.

How 16S- and metagenomic-based models compared at a similar taxonomic level, and how genus and species level predictions compared was examined in order to assess how applicable the modeling strategy could be to different data types. Using paired 16S and shotgun metagenomic sequencing data from Study C, strong agreement between models constructed at the genus level for both 16S and metagenomic data was observed (FIG. 2). Furthermore, robust agreement between predictions at the genus and species levels was observed across metagenomic data sets. Across the in vitro and ex vivo studies, the example results strongly support the use of MCMMs for predicting personalized butyrate and propionate production rates in response to prebiotic, probiotic, and dietary interventions.

In vivo validation via direct measurement of SCFA production is not easily accomplished, due to the rapid consumption of these metabolites by the colonic epithelium and noisy measurements in either stool or serum. However, higher SCFA production rates are known to influence the phenotype of the host in a number of ways, including a reduction in systemic inflammation and improvements in cardiometabolic health. Wastyk et al. found that among 18 individuals given a 10-week high fiber dietary intervention, one third showed an increase in inflammation over the course of the intervention and two thirds showed a decline in systemic markers of inflammation. In the original paper, there was no clear mechanism for explaining these variable immune response groups. The presented example here found that propionate production, a strong inhibitor of inflammation through activation of FFA2 and FFA341,42, was predicted to be significantly lower in individuals who showed the high inflammation response (FIG. 8B-C). The presented example also accessed blood-based clinical labs and microbiome data for a cohort of 2,687 Americans. MCMMs were constructed for this cohort, assuming a standard European diet, and predicted butyrate and propionate production. Butyrate was negatively associated with systemic inflammation, LDL cholesterol, and insulin resistance, blood pressure, and BMI (FIG. 9). These results are consistent with what is known about how butyrate is protective against inflammation, cardiovascular disease, obesity, and metabolic syndrome (FIG. 9B) demonstrating a practical use of the techniques and methods described in the example. Dietary interventions have long been known to elicit variable responses, but a mechanistic framework for predicting this microbiome-mediated heterogeneity has not been available until now.

Given this set of promising associations between SCFA predictions and host phenotypic variation, the example provided demonstrates the potential for leveraging MCMMs for designing precision prebiotic, probiotic, and dietary interventions. Using the Arivale cohort, the example identified two classes of individuals that responded differently to an in silico high-fiber dietary intervention: non-responders and regressors (FIG. 10). Combinatorial interventions were designed that added either a prebiotic or a probiotic to the background diets, to see if rescue of these non-responder and regressor phenotypes could be achieved. Significant heterogeneity in which combinatorial intervention was optimal across individuals from each of these response groups was observed (FIG. 10E). Given that the non-responders had low baseline levels of butyrate production to begin with and did not respond to a high-fiber diet, this result underscores the importance of personalized predictions for those who tend not to respond well to population-scale interventions. These results also suggest that butyrate production in some individuals is limited by composition of the microbiota, indicating that probiotic interventions would be necessary to induce meaningful increases in production.

It will be appreciated that this example had several limitations that should be considered, and that such limitations imply that additional correlations may exist between SCFA and the used of MCMMs to customize nutritional supplementation recommendations. Negative results and failures to identify correlations between SCFA and biomarkers, for instance, may simply be the result of limited data and/or sample sizes. For instance, the example was limited by the availability of high-quality fluxomic data sets for model validation. Sample sizes were limited in the ex vivo fecal studies presented above, due to the cost and difficulty of generating these kinds of data for larger cohorts. Future advances may render such limitations obsolete. Additionally, the human cohort data presented here only provided indirect support for our MCMM predictions (FIGS. 4-5). Furthermore, predictions are dependent on the availability of GEMs. Obtaining large numbers of GEMs that faithfully recapitulate the full metabolic capacities of each organism in a sample is a challenging task. The example used the publicly available AGORA model database. While AGORA models have gone through some degree of manual curation, many of these models are not fully validated and have been shown to include infeasible and missing reactions. Nevertheless, these GEMs work well in the context of butyrate and propionate flux predictions. However, for at least these reasons, it should be appreciated that the example serves only as a demonstration of at least some actions and capabilities of some embodiments of the invention.

The example provided presents an approach for the rational prediction of personalized SCFA production rates from the human gut microbiome, validated using in vitro, ex vivo and in vivo experimental data. Additional analysis demonstrated a clear relationship between SCFA predictions and physiological responses in the host, including lower inflammation and improved cardiometabolic health. SCFA predictions were also significantly associated with variable immune responses to a high fiber dietary intervention. Finally, the example showed how MCMMs could be used to rapidly design and test combinatorial prebiotic, probiotic and dietary interventions in silico for a large human population. Personalized prediction of SCFA production profiles from human gut MCMMs represents an important technological step forward in leveraging computational systems biology for precision nutrition. Mechanistic modeling allows for the translation of the ecological composition of the gut microbiome into concrete, individual-specific metabolic outputs, in response to particular interventions. MCMMs are transparent models that do not require training data, with clear causal and mechanistic explanations behind each prediction. The clinical relevance of these predictions is evident, due to the widespread physiological effects of SCFAs on the human body. A rational framework for engineering the production or consumption rates of these metabolites has broad potential applications in precision nutrition and personalized healthcare.

### Materials and Methods

### In Vitro Culturing

Culturing of the synthetically assembled gut microbial communities is described in Clark et al., 2021. Culturing of ex vivo samples in Study A was done using the methodology described below. Culturing of ex vivo samples in Study B is described in Cantu-Jungles et al., 2021. Culturing of ex vivo samples in Study C was conducted using the methodology described below.

### In Vitro Culturing of Fecal-Derived Microbial Communities (Study A)

Fecal samples were collected in 1200 mL 2-piece specimen collectors (Medline, USA) in the Public Health Science Division of the Fred Hutchinson Cancer Center (IRB Protocol number 5722) and transferred into an large vinyl anaerobic chamber (Coy, USA, 37° C., 5% hydrogen, 20% carbon dioxide, balanced with nitrogen) at the Institute for Systems Biology within 20 minutes of defecation. All further processing and sampling was then run inside the anaerobic chamber. 50 g of fecal material was transferred into sterile 50 oz Filter Whirl-Paks (Nasco, USA) with sterile PBS+0.1% L-cysteine at a 1:2.5 w/v ratio and homogenized with a Stomacher Biomaster (Seward, USA) for 15 minutes. After homogenization, each sample was transferred into three sterile 250 mL serum bottles and another 2.5 parts of PBS+0.1% L-cysteine was added to bring the final dilution to 1:5 in PBS. 87 ug/mL inulin or an equal volume of sterile PBS buffer were added to treatment or control bottles, respectively. Samples were immediately pipetted onto sterile round-bottom 2 mL 96-well plates in triplicates. Baseline samples were aliquoted into sterile 1.5 mL Eppendorf tubes and the plates were covered with Breathe-Easy films (USA Scientific Inc., USA). Plates were incubated for 7 h at 37° C. and gently vortexed every hour within the chamber. Final samples were aliquoted into 1.5 mL Eppendorf tubes at the end of incubation. Baseline and 7 h samples were kept on ice and immediately processed after sampling. 500 uL of each sample were aliquoted for metagenomics and kept frozen at −80° C. before and during transfer to the commercial sequencing service (Diversigen, Inc). The remaining sample was transferred to a table-top centrifuge (Fisher Scientific accuSpin, USA) and spun at 1,500 rpm for 10 minutes. The supernatant was then transferred to collection tubes kept on dry ice and transferred to the commercial metabolomics provider Metabolon, USA, for targeted SCFA quantification.

### In Vitro Culturing of Fecal-Derived Microbial Communities (Study C)

Homogenized fecal samples in this study again underwent anaerobic culturing at 37° C., as described above, but with a shorter culturing time of 4 hours. The slurry was diluted 2.5× in 0.1% L-cysteine PBS buffer solution. Cultures were supplemented with the dietary fibers pectin or inulin to a final concentration of 10 g/L, or a sterile PBS buffer control treatment. Aliquots were taken at 0 h and 4 h and further processed for measurement of SCFA concentrations, which were used to estimate experimental production flux (concentration[4 h]-concentration[0 h]/4 h). SCFA concentrations were measured using GC-FID. Briefly, the pH of the aliquots was adjusted to 2-3 with 1% aqueous sulfuric acid solution, after which they were vortexed for 10 minutes and centrifuged for 10 minutes at 10,000 rpm. 200 μL aliquots of clear supernatant were transferred to vials containing 200 uL of MeCN and 100 uL of a 0.1% v/v 2-methyl pentanoic acid solution. The resulting solutions were analyzed by GC-FID on a Perkin Elmer Clarus 500 equipped with a DB-FFAP column (30 m, 0.250 mm diameter, 0.25 um film) and a flame ionization detector.

### Metagenomic Sequencing and Analysis

For Study A, shallow metagenomic sequencing was performed by the sequencing vendor Diversigen, USA (i.e., their BoosterShot service). In brief, DNA was extracted from the fecal slurries with the DNeasy PowerSoil Pro Kit on a QiaCube HT (Qiagen, Germany) and quantified using the Qiant-iT Picogreen dsDNA Assay (Invitrogen, USA). Library preparation was performed with a proprietary protocol based on the Nextera Library Prep kit (Illumina, USA) and the generated libraries were sequenced on a NovaSeq (Illumina, USA) with a single-end 100 bp protocol. Demultiplexing was performed using Illumina BaseSpace to generate the final FASTQ files used during analysis.

Preprocessing of raw sequencing reads was performed using FASTP. The first 5 bp on the 5′ end of each read were trimmed, and the 3′ end was trimmed using a sliding window quality filter that would trim the read as soon as the average window quality fell below 20. Reads containing ambiguous base calls or with a length of less than 15 bp after trimming were removed from the analysis.

Bacterial species abundances were quantified using Kraken2 v2.0.8 and Bracken v2.2 using the Kraken2 default database which was based on Refseq release 94, retaining only those species with at least 10 assigned reads. The analysis pipeline can be found at https://github.com/Gibbons-Lab/pipelines/tree/master/shallow shotgun, which is hereby incorporated by reference in its entirety.

### Metabolomics

Targeted metabolomics were performed using Metabolon's high-performance liquid chromatography (HPLC)-mass spectrometry (MS) platform, as described before. In brief, fecal supernatants were thawed on ice, proteins were removed using aqueous methanol extraction, and organic solvents were removed with a Turbo Vap (Zymark, USA). Mass spectroscopy was performed using a Waters ACQUITY ultra-performance liquid chromatography (UPLC) and Thermo Scientific Q-Exactive high resolution/accuracy mass spectrometer interfaced with a heated electrospray ionization (HESI-II) source and an Orbitrap mass analyzer operated at 35,000 mass resolution. For targeted metabolomics ultra-pure standards of the desired short-chain fatty acids were used for absolute quantification. Fluxes for individual metabolites were estimated as the rate of change of individual metabolites during the incubation period (concentration[7h]-concentration[0 h]/7h).

### Model Construction

Taxonomic abundance data summarized to the genus level, inferred from 16S amplicon sequencing or shotgun metagenomic sequencing, were used to construct all MCMMs in this analysis using the community-scale metabolic modeling platform MICOM v0.32.3. Models were built using the MICOM build( ) function with a relative abundance threshold of 0.001, omitting taxa that made up less than 0.1% relative abundance. The AGORA database (v1.03) of taxonomic reconstructions summarized to the genus level was used to collect genome-scale metabolic models for taxa present in each model. In silico media were applied to the grow( ) function, defining the bounds for metabolic imports by the MCMM. Medium composition varied between analyses (see Media Construction). Steady state growth rates and fluxes for all samples were then inferred using cooperative tradeoff flux balance analysis (ctFBA). In brief, this is a two-step optimization scheme, where the first step finds the largest possible biomass production rate for the full microbial community and the second step infers taxon-specific growth rates and fluxes, while maintaining community growth within a fraction of the theoretical maximum (i.e., the tradeoff parameter), thus balancing individual growth rates and the community-wide growth rate. For all models in the example, a tradeoff parameter of 0.7 was used. This parameter value was chosen through cooperative tradeoff analysis in MICOM. Multiple parameters were tested, and the highest parameter value (i.e., the value closest to the maximal community growth rate at 1.0) that allowed most (>90%) of taxa to grow was chosen (i.e., 0.7). Predicted growth rates from the simulation were analyzed to validate correct behavior of the models. All models were found to grow with minimum community growth rate of 0.3 h⁻¹. Predicted values for export fluxes of SCFAs were collected from each MCMM using the production_rates( )function, which calculates the overall production from the community that would be accessible to the colonic epithelium.

### Media Construction

Individual media were constructed based on the context of each individual analysis. For the synthetic in vitro cultures conducted by Clark et al. (2021), a defined medium (DM38) was used that supported growth of all taxa used in the experiments, excluding Faecalibacterium prausnitzii. To manually map each component to the Virtual Metabolic Human database, an in silico medium with flux bounds scaled to component concentration was constructed. All metabolites were found in the database. Using the MICOM fix_medium( )function, a minimal set of metabolites necessary for all models to grow to a minimum community growth rate of 0.3 h⁻¹ was added to the medium-here, only iron(III) was added (in silico medium available here: https://github.com/Gibbons-Lab/scfa_predictions/trec/main/media), which is hereby incorporated by reference in its entirety.

To mimic the medium used in ex vivo cultures of fecally-derived microbial communities, a diluted, carbon-stripped version of a standard European diet was used. First, a standard European diet was collected from the Virtual Metabolic Human database (www.vmh.life/#nutrition). Components in the medium which could be imported by the host, as defined by an existing uptake reaction in the Recon3D model, were diluted to 20% of their original flux, to adjust for absorption in the small intestine. Additionally, host-supplied metabolites such as mucins and bile acids were added to the medium. As most carbon sources are consumed in the body and are likely not present in high concentrations in stool, this diet was then algorithmically stripped of carbon sources by removing metabolites with greater than six carbons and no nitrogen, to avoid removing nitrogen sources. Additionally, the remaining metabolites in the medium were diluted to 10% of their original flux, mimicking the nutrient-depleted fecal homogenate. This medium was also augmented using the fix_medium( )function in MICOM. To simulate fiber supplementation, single fiber additions were made to the medium, either pectin (0.75 mmol/gDW*h) or inulin (10.5 mmol/gDW*h). Bounds for fiber supplementation were chosen to balance the carbon content of each, as represented in the model (pectin: 2535 carbons, inulin: 180 carbons).

For in vivo modeling, two diets were used: a high-fiber diet containing high levels of resistant starch, and a standard European diet. Again, both diets were collected from the Virtual Metabolic Human database (www.vmh.life/#nutrition). Each medium was subsequently adjusted to account for absorption in the small intestine by diluting metabolite flux as described previously. Additionally, host-supplied metabolites such as mucins and bile acids were added to the medium, to match the composition of the medium in vivo. Finally, the complete_medium( ) function was again used to augment the medium, as described above.

Prebiotic interventions were designed by supplementing the high-fiber or average European diet with single fiber additions, either pectin or inulin. As before, bounds for fiber addition were set as 0.75 mmol/gDW*h for pectin and 10.5 mmol/gDW*h for inulin.

### Probiotic Intervention

To model a probiotic intervention, 10% relative abundance of the genus Faecalibacterium, a known butyrate-producing taxon, was added to the MCMMs by adding a pan-genus model of the taxon derived from the AGORA database version 1.03. Measured taxonomic abundances were scaled to 90% of their initial values, after which Faecalibacterium was artificially added to the model.

### External Data Collection

Data containing taxonomic abundance, optical density, and endpoint butyrate concentration for synthetically-constructed in vitro microbial cultures were collected from Clark et al. (2021). Endpoint taxonomic abundance data, calculated from fractional read counts collected via 16S amplicon sequencing, was used to construct individual MCMMs for each co-culture (see Model Construction). Resulting models ranged in taxonomic richness from 1 to 25 taxa.

From a second study by Cantu-Jungles et al. (2021) (ex vivo Study B), preprocessed taxonomic abundance and SCFA metabolomics data was collected. Homogenized fecal samples in this study underwent a similar culturing process, with a culturing time of 24 hours. Cultures were supplemented with the dietary fiber pectin, or a PBS control. Initial and endpoint metabolomic SCFA measurements were used to estimate experimental production flux (concentration[24 h]-concentration[Oh]/24 h). Taxonomic abundance data was used to construct MCMMs for each individual (see Model Construction).

Data from a third (Study C) was collected from the Pharmaceutical Biochemistry Group at the University of Geneva, Switzerland, under study protocol 2019-00632, containing sequencing data in FASTQ format and targeted metabolomics SCFA measurements.

Data was collected from Wastyk, et al 2021, which provided 16S amplicon sequencing data at 9 timepoints spanning 14 weeks, along with immunological phenotyping, for 18 participants undergoing a high-fiber dietary intervention. Only 7 timepoints spanning 10 weeks were included in subsequent analysis, as the last 2 timepoints were taken after the conclusion of the dietary intervention. MCMMs were constructed for each participant at each timepoint at the genus level (see Model Construction). Mean total butyrate and propionate production, as well as acetate production, were compared between immune response groups.

De-identified data was obtained from a former scientific wellness program run by Arivale, Inc. (Seattle, WA). Arivale closed its operations in 2019. Taxonomic abundances, inferred from 16S amplicon sequencing data, for 2,687 research-consenting individuals were collected and used to construct MCMMs. 128 paired blood-based clinical chemistries taken within 30 days of fecal sampling were also collected and used to find associations between MCMM SCFA predictions on a standard European diet and clinical markers.

### Statistical Analysis

Statistical analysis was performed using SciPy (v1.9.1) and statsmodels (v0.14.0) in Python (v3.8.13). Pearson correlation coefficients and p-values were calculated between measured and predicted SCFA production fluxes in in vitro cultures, as well as for predicted SCFA production fluxes across timepoints for an in vivo high-fiber intervention. Significance in overall SCFA production between immune response groups in the high-fiber intervention was determined by pairwise Mann-Whitney U test for butyrate+propionate production and for acetate production. Association of MCMM-predicted SCFA production flux with paired blood-based clinical labs was tested using OLS regression, adjusting for age, sex, microbiome sequencing vendor, and clinical lab vendor, and tested for significance by two-sided Wald test. BMI was not included as a confounder in the analysis because it was itself negatively correlated with butyrate production. Multiple comparison correction for p-values was done using the Benjamini-Hochberg method for adjusting the False Discovery Rate (FDR). Comparison of butyrate production between dietary interventions was tested using paired Student's t-tests. In all analyses, significance was considered at the p<0.05 threshold.

### Data, Software, and Code Availability

Code used to run analysis and create figures for this manuscript can be found at https://github.com/Gibbons-Lab/scfa predictions, which is hereby incorporated by reference in its entirety for all purposes.

Processed data for synthetically constructed cultures can be found at https://github.com/RyanLincolnClark/DesignSyntheticGutMicrobiomeAssemblyFunction, which is hereby incorporated by reference in its entirety for all purposes. Raw sequencing data can be found at https://doi.org/10.5281/zenodo.4642238, which is hereby incorporated by reference in its entirety for all purposes.

Raw sequencing data for Study A can be found in the NCBI SRA under accession number PRJNA937304, which is hereby incorporated by reference in its entirety for all purposes.

Processed data for ex vivo Study B can be found at https://github.com/ThaisaJungles/fiber specificity, which is hereby incorporated by reference in its entirety for all purposes. Raw sequencing data can be found in the NCBI SRA under accession number PRJNA640404, which is hereby incorporated by reference in its entirety for all purposes.

Each of the following references are hereby incorporated by reference in its entirety for all purposes:
1. Oliphant, K. & Allen-Vercoe, E. Macronutrient metabolism by the human gut microbiome: major fermentation by-products and their impact on host health.Microbiome7, 91 (2019).
2. Rackerby, B., Van De Grift, D., Kim, J. H. & Park, S. H. Effects of Diet on Human Gut Microbiome and Subsequent Influence on Host Physiology and Metabolism. Gut Microbiome and Its Impact on Health and Diseases63-84 Preprint at https://doi.org/10.1007/978-3-030-47384-6_3(2020).
3. Tomasova, L., Grman, M., Ondrias, K. & Ufnal, M. The impact of gut microbiota metabolites on cellular bioenergetics and cardiometabolic health.Nutr. Metab.18, 72 (2021).
4. Glotfelty, L. G., Wong, A. C. & Levy, M. Small molecules, big effects: microbial metabolites in intestinal immunity. Am. J. Physiol. Gastrointest. Liver Physiol.318, G907-G911 (2020).
5. Donia, M. S. & Fischbach, M. A. HUMAN MICROBIOTA. Small molecules from the human microbiota.Science349, 1254766 (2015).
6. Diener, C. et al.Genome-microbiome interplay provides insight into the determinants of the human blood metabolome. Nat Metab4, 1560-1572 (2022).
7. Ríos-Covian, D.et al. Intestinal Short Chain Fatty Acids and their Link with Diet and Human Health.Front. Microbiol.7, 185 (2016).
8. Nogal, A., Valdes, A. M. & Menni, C. The role of short-chain fatty acids in the interplay between gut microbiota and diet in cardio-metabolic health. Gut Microbes13, 1-24 (2021).
9. Silva, Y. P., Bernardi, A. & Frozza, R. L. The Role of Short-Chain Fatty Acids From Gut Microbiota in Gut-Brain Communication.Frontiers in Endocrinologyvol. 11 Preprint at https://doi.org/10.3389/fendo.2020.00025(2020).
10. Morrison, D. J. & Preston, T. Formation of short chain fatty acids by the gut microbiota and their impact on human metabolism.Gut Microbes7, 189-200 (2016).
11. Cong, J., Zhou, P. & Zhang, R. Intestinal Microbiota-Derived Short Chain Fatty Acids in Host Health and Disease. Nutrients14, (2022).
12. Tan, J.et al. The role of short-chain fatty acids in health and disease. Adv. Immunol. 121, 91-119 (2014).
13. Mortensen, P. B. & Clausen, M. R. Short-chain fatty acids in the human colon: relation to gastrointestinal health and disease.Scand. J. Gastroenterol. Suppl.216, 132-148 (1996).
14. Cantu-Jungles, T. M.et al.Dietary Fiber Hierarchical Specificity: the Missing Link for Predictable and Strong Shifts in Gut Bacterial Communities.MBio12, e0102821 (2021).
15. Healey, G. R., Murphy, R., Brough, L., Butts, C. A. & Coad, J. Interindividual variability in gut microbiota and host response to dietary interventions.Nutr. Rev.75, 1059-1080 (2017).
16. Boets, E.et al.Quantification of in Vivo Colonic Short Chain Fatty Acid Production from Inulin.Nutrients7, 8916-8929 (2015).
17. Diener, C., Gibbons, S. M. & Resendis-Antonio, O. MICOM: Metagenome-Scale Modeling To Infer Metabolic Interactions in the Gut Microbiota.mSystems5, (2020).
18. van Deuren, T., Blaak, E. E. & Canfora, E. E. Butyrate to combat obesity and obesity-associated metabolic disorders: Current status and future implications for therapeutic use.Obes. Rev.23, e13498 (2022).
19. Zeevi, D. et al. Personalized Nutrition by Prediction of Glycemic Responses. Cell163, 1079-1094 (2015).
20. Rein, M. et al. Effects of personalized diets by prediction of glycemic responses on glycemic control and metabolic health in newly diagnosed T2DM: a randomized dietary intervention pilot trial.BMC Med.20, 56 (2022).
21. Gibbons, S. M.et al. Perspective: Leveraging the Gut Microbiota to Predict Personalized Responses to Dietary, Prebiotic, and Probiotic Interventions. Adv. Nutr. 13, 1450-1461 (2022).
22. Heinken, A. et al.Genome-scale metabolic reconstruction of 7,302 human microorganisms for personalized medicine. Nat. Biotechnol.(2023) doi:10.1038/s41587-022-01628-0.
23. Abdill, R. J., Adamowicz, E. M. & Blekhman, R. Public human microbiome data are dominated by highly developed countries. PLOS Biol. 20, e3001536 (2022).
24. Magnusdottir, S. et al. Generation of genome-scale metabolic reconstructions for 773 members of the human gut microbiota. Nat. Biotechnol. 35, 81-89 (2017).
25. Clark, R. L. et al. Design of synthetic human gut microbiome assembly and butyrate production. Nat. Commun. 12, 3254 (2021).
26. Wastyk, H. C.et al. Gut-microbiota-targeted diets modulate human immune status. Cell184, 4137-4153.e14 (2021).
27. Manor, O. et al. Health and disease markers correlate with gut microbiome composition across thousands of people. Nat. Commun. 11, 5206 (2020).
28. Heinken, A. et al. AGORA2: Large scale reconstruction of the microbiome highlights wide-spread drug-metabolising capacities.bioRxiv2020.11.09.375451 (2020) doi:10.1101/2020.11.09.375451.
29. Valgepea, K. et al. Systems biology approach reveals that overflow metabolism of acetate in Escherichia coli is triggered by carbon catabolite repression of acetyl-CoA synthetase.BMC Syst. Biol. 4, 166 (2010).
30. Wolfe, A. J. The acetate switch. Microbiol. Mol. Biol. Rev.69, 12-50 (2005).
31. Li, M. et al. Pro- and anti-inflammatory effects of short chain fatty acids on immune and endothelial cells. Eur. J. Pharmacol. 831, 52-59 (2018).
32. Arifuzzaman, M.et al. Inulin fibre promotes microbiota-derived bile acids and type 2 inflammation. Nature611, 578-584 (2022).
33. Sproston, N. R. & Ashworth, J. J. Role of C-Reactive Protein at Sites of Inflammation and Infection. Front. Immunol.9, 754 (2018).
34. Amiri, P. et al. Role of Butyrate, a Gut Microbiota Derived Metabolite, in Cardiovascular Diseases: A comprehensive narrative review.Front. Pharmacol.12, 837509 (2021).
35. Vinolo, M. A. R., Rodrigues, H. G., Nachbar, R. T. & Curi, R. Regulation of inflammation by short chain fatty acids.Nutrients3, 858-876 (2011).
36. Tedelind, S., Westberg, F., Kjerrulf, M. & Vidal, A. Anti-inflammatory properties of the short-chain fatty acids acetate and propionate: a study with relevance to inflammatory bowel disease. World J. Gastroenterol.13, 2826-2832 (2007).
37. Gurry, T., Nguyen, L. T. T., Yu, X. & Alm, E. J. Functional heterogeneity in the fermentation capabilities of the healthy human gut microbiota.PLOS One16, e0254004 (2021).
38. Gasaly, N., de Vos, P. & Hermoso, M. A. Impact of Bacterial Metabolites on Gut Barrier Function and Host Immunity: A Focus on Bacterial Metabolism and Its Relevance for Intestinal Inflammation.Front. Immunol. 12, 658354 (2021).
39. Agus, A., Clément, K. & Sokol, H. Gut microbiota-derived metabolites as central regulators in metabolic disorders.Gut70, 1174-1182 (2021).
40. Chen, S., Zhou, Y., Chen, Y. & Gu, J. fastp: an ultra-fast all-in-one FASTQ preprocessor.Bioinformatics34, 1884-1890 (2018).
41. Wood, D. E., Lu, J. & Langmead, B. Improved metagenomic analysis with Kraken 2.Genome Biol.20, 257 (2019).
42. Lu, J., Breitwieser, F. P., Thielen, P. & Salzberg, S. L. Bracken: estimating species abundance in metagenomics data.PeerJ Comput. Sci.3, e104 (2017).
43. Gauglitz, J. M.et al.Enhancing untargeted metabolomics using metadata-based source annotation.Nat. Biotechnol.40, 1774-1779 (2022).
44. Elmadfa, I. Österreichischer Ernährungsbericht 2012.1, (2012).
45. Brunk, E.et al.Recon3D enables a three-dimensional view of gene variation in human metabolism.Nat. Biotechnol.36, 272-281 (2018).
46. Waldmann, A., Koschizke, J. W., Leitzmann, C. & Hahn, A. Dietary intakes and lifestyle factors of a vegan population in Germany: results from the German Vegan Study. Eur. J. Clin. Nutr.57, 947-955 (2003).
47. Zhou, L. et al. Faecalibacterium prausnitzii Produces Butyrate to Maintain Th17/Treg Balance and to Ameliorate Colorectal Colitis by Inhibiting Histone Deacetylase 1. Inflamm. Bowel Dis.24, 1926-1940 (2018).
48. Coppola, S., Avagliano, C., Calignano, A. & Berni Canani, R. The Protective Role of Butyrate against Obesity and Obesity-Related Diseases.Molecules26, (2021).
49. Benjamini, Y. & Hochberg, Y. Controlling the false discovery rate: A practical and powerful approach to multiple testing.J. R. Stat. Soc.57, 289-300 (1995).

What is claimed is:

1. A computer-implemented method of simulating growth in a gut microbiome taxon model for determining a supplemental intervention of a subject, the computer-implemented method comprising:
(a) accessing measured taxon and abundance data of a gut microbiome sample of a subject;
(b) generating a plurality of flux balance analysis (FBA) microbial community-scale metabolic models (MCMMs) of the gut microbiome of the subject, each MCMM constrained by a different background diet, a plurality of genome-scale metabolic models (GEMs) of the measured taxon of the subject, and one or more supplemental interventions comprising:
(i) a probiotic intervention comprising a probiotic taxon added to the measured taxon,
(ii) a prebiotic intervention comprising a non-digestible substrate promoting growth of a beneficial microorganism added to the background diet, or
(iii) a combination thereof;
wherein the probiotic intervention is added at one or more different doses to the MCMM to determine a response to the probiotic intervention, and wherein the prebiotic intervention is added at one or more different doses to the background diet to determine a response to the prebiotic intervention; and
(c) simulating growth in each of the MCMMs so as to predict metabolic productions from addition of the one or more supplemental interventions to the different background diets in the subject.

2. The method of claim 1, wherein the different background diets are selected from (i) a high-fiber diet such as a vegan high-fiber diet rich in resistant starch or a standard Mediterranean diet, (ii) a low fiber diet such as a standard European diet or a standard American diet, and (iii) a personalized diet.

3. The method of claim 1, wherein the supplemental intervention is the combination of the prebiotic intervention and the probiotic intervention.

4. The method of claim 1, wherein the one or more supplemental interventions is absent in the subject before any addition of the one or more supplemental interventions.

5. The method of claim 1, wherein the growth is simulated at increasing increments of the doses of the one or more supplemental interventions so as to generate a plurality of metabolic productions characterizing a dose escalation of the one or more supplemental interventions.

6. The method of claim 5, where the plurality of metabolic productions includes a metabolic production for short chain fatty acid (SCFA) production comprising, or selected from, butyrate production, propionate production, acetate production, or a combination thereof, and wherein the simulation is configured to classify the subject as a responder, non-responder, or regressor based on simulated SCFA production in response to the background diet, the one or more supplemental intervention, or a combination thereof.

7. The method of claim 6, further comprising repeating steps (b) and (c) for a classification comprising, or selected from, the non-responder and the regressor, with one or more additional supplemental interventions.

8. The method of claim 1, further comprising:
(d) ranking the one or more supplemental interventions according to the different background diets and the predicted metabolic production; and
(e) generating a gut health management recommendation comprising part or all of the ranking.

9. The method of claim 8, wherein the ranking is a heatmap ranking.

10. The method of claim 8, wherein generating the gut health management recommendation further comprises:
(i) mapping the predicted metabolic production of the subject to metabolic production of a reference population, and optionally, clinical phenotypes associated with metabolic production of the subject, the reference population, or a combination thereof, and
(ii) generating a distribution of the metabolic production of the reference population and embedding the predicted metabolic production of the subject into a context of the distribution; and
(iii) generating a comparative metric using the predicted metabolic production of the subject and the distribution, wherein the comparative metric represents whether or where the predicted metabolic production of the subject falls within the distribution.

11. The method of claim 10, wherein the mapping and distribution includes the clinical phenotypes associated with metabolic production of the subject, the reference population, or a combination thereof, and wherein the clinical phenotypes are blood-based clinical labs and health markers.

12. The method of claim 11, wherein the clinical phenotypes are cardiometabolic and immunological health markers.

13. The method of claim 12, wherein the cardiometabolic and immunological health markers are associated with butyrate production having (i) significant positive associations with blood-derived markers comprising, or selected from, adiponectin, chloride, and high density lipoprotein (HDL) cholesterol, and (ii) significant negative associations with C-reactive protein (CRP), low-density lipoprotein (LDL), and blood pressure.

14. The method of claim 12, wherein the cardiometabolic and immunological health markers associated with butyrate production comprise, or are selected from, absolute monocytes count, alanine transaminase, arachidonic acid, blood pressure, glucose, high sensitivity CRP, LDL, LDL cholesterol, LDL small particle number, LP-IR scores, mean corpuscular hemoglobin concentration, oxidized LDL, platelets, triglyceride/HDL ratio, triglycerides, uric acid, and zinc.

15. A gut health intervention identification system comprising:
one or more processors; and
memory coupled to the one or more processors, wherein the memory comprises computer-executable instructions causing the one or more processors to perform a process comprising:
(a) receiving butyrate production data of:
(i) a gut microbiome of a subject simulated on a plurality of different background diets with and without one or more supplemental interventions, the one or more supplemental interventions comprising, or selected from, a prebiotic intervention, a probiotic intervention, or a combination thereof, and
(ii) a plurality of gut microbiomes of a reference population comprising generally healthy individuals each individually simulated for butyrate production on essentially the same background diets as the subject, and optionally, essentially the same supplemental interventions as the subject;
(b) generating, for each of the plurality of different background diets, a distribution based on butyrate production data of the subject and the reference population associated with the background diet;
(c) generating, for each of the plurality of different background diets, a comparative metric using the distribution for the background diet and the butyrate production data of the subject simulated on the background diet; and
(d) identifying, based on the comparative metrics, a particular intervention to recommend for the subject, where the particular intervention includes a particular background diet, one or more particular supplemental interventions, or a combination thereof.

16. The system of claim 15, wherein the particular intervention is predicted to result in a butyrate production in the subject that meets or exceeds a minimum healthy butyrate production threshold of the reference population simulated for butyrate production on essentially the same background diet as the subject.

17. The system of claim 16, wherein the minimum healthy butyrate production threshold is a cutoff between lower and inter quartiles of the reference population for butyrate production.

18. The system of claim 15, further generating a gut health report embedding the butyrate production data of the subject into a context of the distribution of the butyrate production of the reference population for a given background diet of the plurality of different background diets, the gut health report identifying the particular intervention.

19. The system of claim 15, wherein the identifying the particular intervention comprises ranking the plurality of background diets based on the comparative metrics.

20. The system of claim 19, wherein the ranking further comprises a clinical phenotype associated with the ranking, and wherein the process further includes generating a gut health management recommendation based on part or all of the ranking.

21. The system of claim 15, further comprising:
generating associations between clinical phenotypes and butyrate production of the reference population, and
generating a comparative metric using the associations, wherein the comparative metric represents whether the predicted butyrate production of the subject is positively or negatively associated with the clinical phenotype.

22. The system of claim 21, wherein the clinical phenotype comprises, or is selected from, cardiometabolic and immunological health markers.

23. The system of claim 22, wherein the cardiometabolic and immunological health markers are associated with butyrate production, the butyrate production having (i) significant positive associations with blood-derived markers comprising, or selected from, adiponectin, chloride, and high density lipoprotein (HDL) cholesterol, and (ii) significant negative associations with C-reactive protein (CRP), low-density lipoprotein (LDL), and blood pressure.

24. The system of claim 23, wherein the cardiometabolic and immunological health markers associated with butyrate production comprise: absolute monocytes count, alanine transaminase, arachidonic acid, blood pressure, glucose, high sensitivity CRP, LDL, LDL cholesterol, LDL small particle number, LP-IR scores, mean corpuscular hemoglobin concentration, oxidized LDL, platelets, triglyceride/HDL ratio, triglycerides, uric acid, or zinc.

25. The system of claim 15, wherein the plurality of background diets comprises a high-fiber diet such as a vegan high-fiber diet rich in resistant starch or a standard Mediterranean diet, a low fiber diet such as a standard European diet or a standard American diet, and a personalized diet.

26. The system of claim 15, further comprising, for each of the plurality of different background diets with and without one or more supplemental interventions:
generating a classification that predicts whether the subject will be a responder, non-responder, or regressor to the background diet with or without the one or more supplemental inventions, wherein the responder exhibits essentially an increase in butyrate production, the non-responder exhibits essentially no change in butyrate production, and the regressor exhibits essentially a decrease in butyrate production.
